(19) United States
(12) Patent Application Publication
(10) Pub. No.: US 2026/0335542 A1
(43) Pub. Date: Oct. 29, 2026

(54) SYRINGE PUMP DRUG DELIVERY DEVICE

(71) Applicant: Tandem Diabetes Care, Inc., San Diego, CA (US)

(72) Inventors: Adam Evard, San Diego, CA (US); Edward Ho, San Diego, CA (US)

(21) Appl. No.: 19/119,554

(22) PCT Filed: Oct. 9, 2023
(86) PCT No.: PCT/US2023/076355

§ 371 Date: Apr. 9, 2025

Related U.S. Application Data
(60) Provisional application No. 63/414,689, filed on Oct. 10, 2022.

(51) Int. Cl. A61M 5/145; A61M 5/142

(52) U.S. Cl. CPC A61M 5/1452; A61M 5/14248; A61M 2205/02

(57) ABSTRACT

Disclosed herein are apparatuses and methods relating to an ambulatory infusion pumps having a syringe pumping mechanism. Pumps utilizing the pump mechanism disclosed herein can have a smaller form factor than existing syringe pumps by configuring the drive mechanism such that the leadscrew that drives the syringe plunger is disposed within the medicament reservoir.

This application is being filed on Oct. 9, 2023, as a PCT International Patent application and claims the benefit of and priority to U.S. provisional patent application Ser. No. 63/414,689, filed Oct. 10, 2022, the entire disclosure of which is incorporated by reference herein in its entirety.

### TECHNICAL FIELD

The present disclosure relates generally to ambulatory infusion pumps and, more particularly, to a user-wearable pump for delivering medicament such as insulin to a patient.

### BACKGROUND

There are a wide variety of medical treatments that include the administration of a therapeutic fluid in precise, known amounts at predetermined intervals. Devices and methods exist that are directed to the delivery of such fluids, which may be liquids or gases, are known in the art.

One category of such fluid delivery devices includes insulin injecting pumps developed for administering insulin to patients afflicted with type I, or in some cases, type II diabetes. Some insulin injecting pumps are configured as portable or ambulatory infusion devices can provide continuous subcutaneous insulin injection and/or infusion therapy as an alternative to multiple daily injections of insulin via a syringe or an injector pen. Such ambulatory infusion pumps are worn by the user and may use replaceable cartridges. In some embodiments, these pumps may also deliver medicaments other than, or in addition to, insulin, such as glucagon, pramlintide, and the like. Examples of such pumps and various features associated therewith include those disclosed in U.S. Patent Publication Nos. 2013/0324928 and 2013/0053816 and U.S. Pat. Nos. 8,287,495; 8,573,027; 8,986,253; and 9,381,297, each of which is incorporated herein by reference in its entirety.

User-wearable infusion pumps can employ various different pumping mechanisms for delivering insulin and/or other medicaments from a reservoir to the user. One common pumping mechanism employs a syringe that forces medicament out of the reservoir. Typically, a plunger within the reservoir can be driven by a leadscrew interfaced with a motor, such that when the motor is actuated the leadscrew causes the plunger to be advanced within the reservoir to cause medicament to be delivered out of an outlet in the reservoir.

One type of pump that has been developed is a patch pump, or micro pump. Patch pumps generally are small pumps, typically ambulatory, that are carried directly on the skin under the user's clothing. Many such pumps are situated directly on the infusion site such that no tubing is required to deliver the insulin and/or other medicament to the patient. Other patch pumps can be positioned on the patient's body with a short length of tubing extending to a nearby infusion site. Some patch pumps can be at least in part disposable, meant to be worn for a period of time such as, e.g., a day or two, and then discarded and replaced by a new patch pump. Other patch pump designs contemplate a disposable component, such as a cartridge that contains medicament, and a reusable or durable component. In such configurations, the disposable and durable components may be joined together by the patient or caregiver in preparation for delivery of the medicament. Many users would like an infusion pump such as a patch pump to be as small as possible in order to be more comfortable and be carried more discreetly on the user's body. However, drive mechanisms such syringe mechanisms described above can place constraints on how small a pump such as a patch pump can be made.

### SUMMARY

Disclosed herein are apparatuses and methods relating to an ambulatory infusion pumps having a syringe pumping mechanism. Pumps utilizing the pump mechanism disclosed herein can have a smaller form factor than existing syringe pumps by configuring the drive mechanism such that the leadscrew that drives the syringe plunger is disposed within the medicament reservoir.

In an embodiment, an ambulatory infusion pump can include a reservoir configured to contain a medicament and a leadscrew having a threaded exterior surface extending through the reservoir. A syringe plunger can be disposed within the reservoir and have a threaded interior surface interfacing with the threaded exterior surface of the leadscrew such that rotation of the leadscrew causes linear movement of the syringe plunger through the reservoir to cause the medicament to be delivered from the reservoir to a user.

In an embodiment, an ambulatory infusion pump can include a reservoir configured to contain a medicament and a leadscrew extending through the reservoir. A syringe plunger can be disposed within the reservoir and have an opening through which the leadscrew is extended. Rotation of the leadscrew can cause linear movement of the syringe plunger along the leadscrew through the reservoir to cause the medicament to be delivered from the reservoir to a user

The above summary is not intended to describe each illustrated embodiment or every implementation of the subject matter hereof. The figures and the detailed description that follow more particularly exemplify various embodiments.

### BRIEF DESCRIPTION OF THE DRAWINGS

The invention may be more completely understood in consideration of the following detailed description of various embodiments of the invention in connection with the accompanying drawings, in which:

FIGS. 1A-1B schematically depict a portion of a leadscrew driven infusion pump according to the prior art.

FIGS. 2A-2B schematically depict a portion of a user-wearable infusion pump according to the present disclosure.

FIGS. 3A-3C depict a user-wearable infusion pump according to the present disclosure.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

The following detailed description should be read with reference to the drawings in which similar elements in different drawings are numbered the same. The drawings, which are not necessarily to scale, depict illustrative embodiments and are not intended to limit the scope of the disclosure.

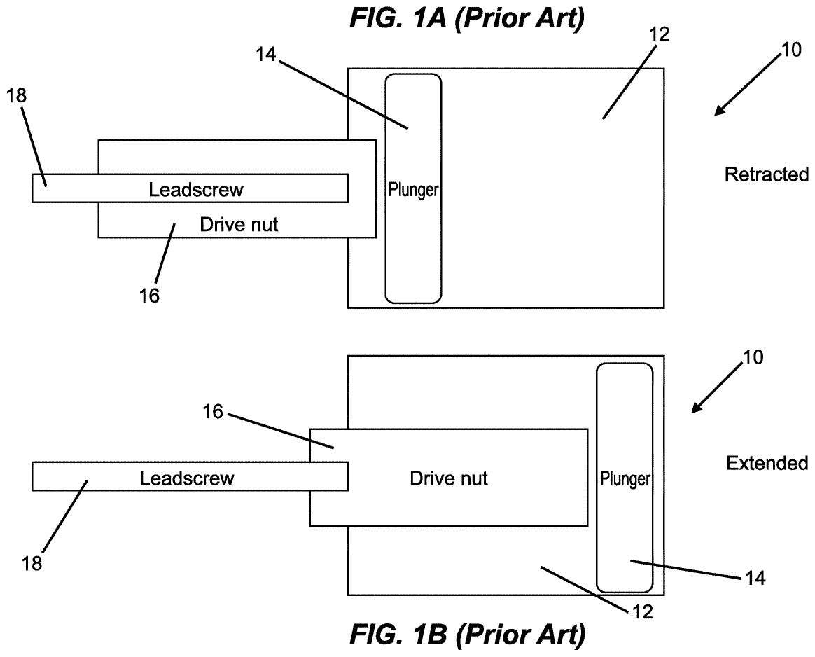

FIGS. 1A-1B schematically depict a portion of a leadscrew driven user-wearable infusion pump 10. Although infusion pump 10 would include a number of additional components, such as, for example, a motor, processor, housing, etc., only the syringe components are depicted for sake of clarity. Pump 10 includes a reservoir 12 configured to contain a medicament with a plunger 14 disposed therein. Plunger 14 is attached to a drive nut 16 that interfaces with a leadscrew 18. In operation, when the motor is actuated to rotate the lead screw 18, the drive nut 16, which is constrained from rotating, is linearly advanced to move the plunger 14 within the reservoir 12 to deliver medicament from the reservoir. FIG. 1A depicts the plunger 14 in the retracted position and FIG. 1B depicts the plunger 14 in the fully extended position (i.e., the reservoir is empty because all medicament has been delivered). As can be seen in these Figures, the length of the overall system has two be more than twice the length of the reservoir in order to deliver all of the fluid from the reservoir due to the overlapping and telescoping nature of the components of the system. This constraint provides a significant challenge in miniaturizing pump delivery systems.

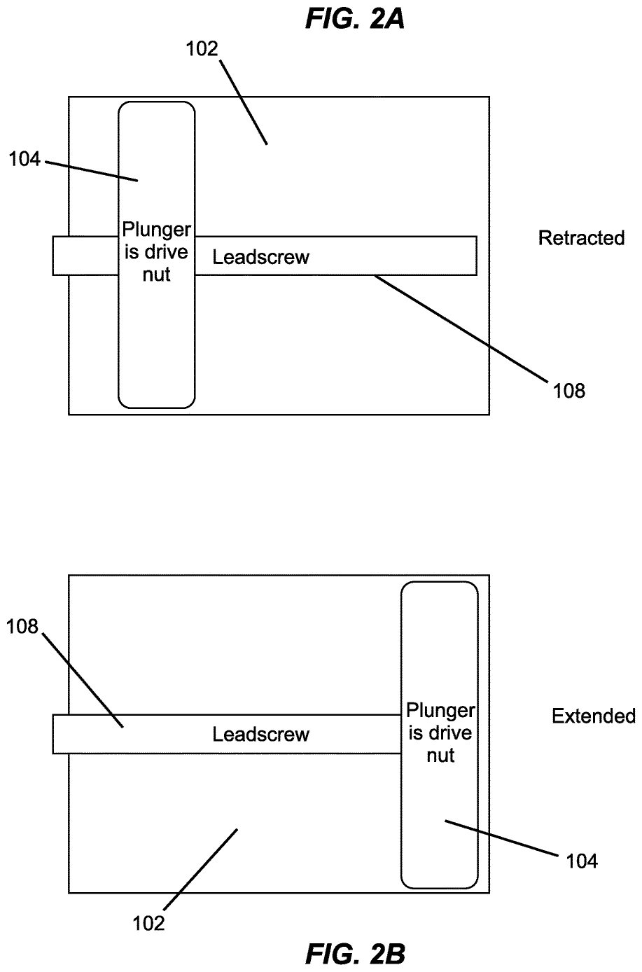

Referring now to FIGS. 2A-2B, the syringe components of a user-wearable infusion pump according to the present disclosure are schematically depicted. This embodiment solves the aforementioned problem regarding the required length of the system by positioning the leadscrew 108 within the reservoir 102. In this embodiment, the leadscrew 108 is directly interfaced with the plunger 104 such that the leadscrew 108 extends through an opening in the plunger 104 with corresponding threads on the exterior of the leadscrew and the interior of the plunger interfacing with one another. When the leadscrew 108 is rotated, the plunger 104 moves along the leadscrew 108 through the reservoir 102 to cause medicament to be delivered. This design therefore removes the drive nut 16 of the prior embodiment and instead provides threading on the plunger 104 that functions similarly to the drive nut 16. Removal of the separate drive nut components and incorporation of the threads onto the plunger enables the leadscrew 108 to be contained within the reservoir to greatly reduced the required length of the drive system. As can be seen in comparing FIGS. 2A-2B with FIGS. 1A-1B, this design enables a pump with a significantly smaller footprint to be manufactured.

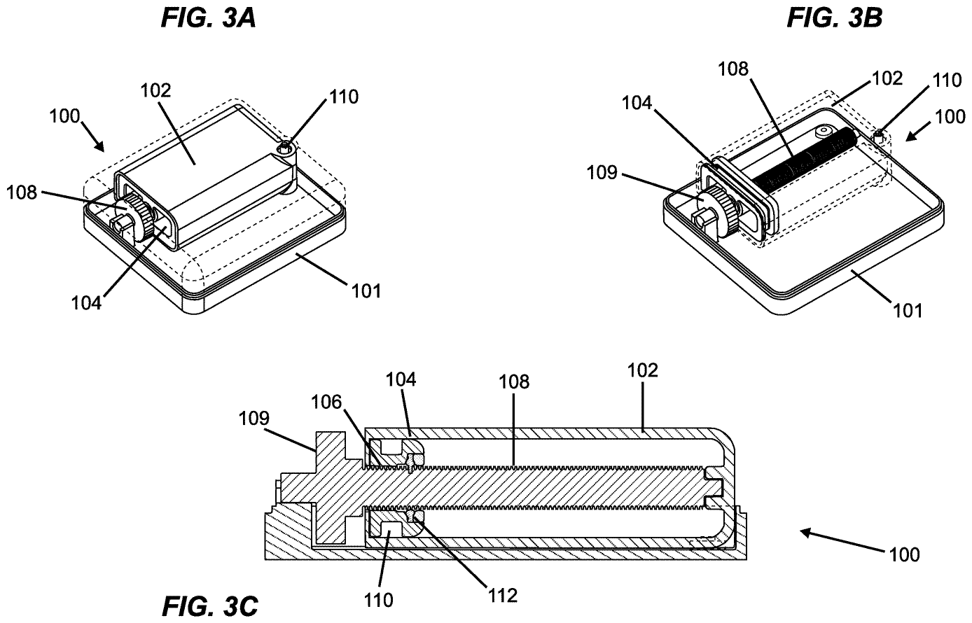

FIGS. 3A-3C depict a leadscrew design such as in FIGS. 2A-2B being employed with a user-wearable infusion pump 100, which may be configured as a disposable patch pump, according to an embodiment. Pump 100 includes a housing 101 containing a reservoir 102 configured to contain a medicament and a leadscrew 108 configured to be rotated by a motor to drive a plunger 104 through the reservoir to deliver the medicament to the user. A portion 109 of leadscrew that interfaces with the motor to enact rotation of the leadscrew is disposed outside of the reservoir 102 within housing 101. As the plunger 104 advances distally through the reservoir from the proximal starting position of a fully filled reservoir 102 depicted in these figures, the plunger 104 causes medicament from the reservoir to be expelled out of a port 110 near at a distal end of the reservoir opposite from the starting position of the plunger.

As noted above, plunger 104 can be provided with threading 106 to enable the drive nut of typical syringe drive systems to be eliminated. Plunger 104 therefore moves directly along the threads of leadscrew 108 such that the leadscrew can be disposed within the reservoir 102. Referring in particular to FIG. 3B, the rectangular shape of plunger 104 disposed within the matching interior cross-section of the reservoir 102 prevents the plunger 104 from rotating when the leadscrew 108 is rotated, such that rotation of the leadscrew causes linear movement of the plunger 104 through the reservoir 102. A syringe seal 110 can extend around the interface between plunger 104 and reservoir 102 and a thread seal 112 around the interface between plunger 104 and leadscrew 108 to prevent leakage of fluid. Although the embodiment of pump 100 depicted in FIGS. 3A-3C pertains to a patch pump having a self-contained reservoir, it should be understood that the same components could be employed in a pump system having a disposable or refillable cartridge containing a reservoir that interfaces with a reusable pump body including the drive mechanism. In embodiments, leadscrew 108 can comprise stainless steel, plastic, or other material that will not contaminate the medicament with which it is in contact with in the reservoir. Additional and/or alternative components of such an infusion pump system can be found in U.S. Patent Publication Nos. 2011/0144586, 2016/0339172 and 2017/0049957, PCT Publication No. WO 2023/129651 and U.S. Pat. No. 10,279,106, each of which is incorporated herein by reference.

In embodiments, an ambulatory infusion pump can include a reservoir configured to contain a medicament and a leadscrew having a threaded exterior surface extending through the reservoir. A syringe plunger can be disposed within the reservoir and have a threaded interior surface interfacing with the threaded exterior surface of the leadscrew such that rotation of the leadscrew causes linear movement of the syringe plunger through the reservoir to cause the medicament to be delivered from the reservoir to a user.

In some embodiments, the syringe plunger has an outer perimeter shape generally matching a shape of an inner cross-section of the reservoir.

In some embodiments, the syringe plunger provides a seal against the inner cross-section of the reservoir to prevent medicament from leaking proximally passed the syringe plunger.

In some embodiments the pump further includes a sealing element extending around an interface between the syringe plunger and the inner cross-section of the reservoir.

In some embodiments, the pump further includes a sealing element disposed around an interface between the leadscrew and the syringe plunger.

In some embodiments, the pump further includes a motor configured to cause the leadscrew to rotate.

In some embodiments, the syringe plunger does not rotate when the leadscrew rotates.

In some embodiments, a portion of the leadscrew that interfaces with the motor is disposed outside of the reservoir.

In some embodiments, the leadscrew is formed of a material that will not contaminate the medicament.

In some embodiments, the pump further includes an outlet port at a distal end of the reservoir, and wherein the syringe plunger causes medicament to be expelled out of the outlet port as the syringe plunger is advanced distally along the leadscrew.

In embodiments, an ambulatory infusion pump can include a reservoir configured to contain a medicament and a leadscrew extending through the reservoir. A syringe plunger can be disposed within the reservoir and have an opening through which the leadscrew is extended. Rotation of the leadscrew can cause linear movement of the syringe plunger along the leadscrew through the reservoir to cause the medicament to be delivered from the reservoir to a user.

In some embodiments, the syringe plunger has an outer perimeter shape generally matching a shape of an inner cross-section of the reservoir.

In some embodiments, the syringe plunger provides a seal against the inner cross-section of the reservoir to prevent medicament from leaking proximally passed the syringe plunger.

In some embodiments, the pump further includes a sealing element extending around an interface between the syringe plunger and the inner cross-section of the reservoir.

In some embodiments, the pump further includes a sealing element disposed around an interface between the leadscrew and the syringe plunger.

In some embodiments, the pump further includes a motor configured to cause the leadscrew to rotate.

In some embodiments, the syringe plunger does not rotate when the leadscrew rotates.

In some embodiments, a portion of the leadscrew that interfaces with the motor is disposed outside of the reservoir.

In some embodiments, the leadscrew is formed of a material that will not contaminate the medicament.

In some embodiments, the further includes an outlet port at a distal end of the reservoir, and the syringe plunger causes medicament to be expelled out of the outlet port as the syringe plunger is advanced distally along the leadscrew.

Although embodiments described herein may be discussed in the context of the controlled delivery of insulin, delivery of other medicaments, singly or in combination with one another or with insulin, including, for example, glucagon, pramlintide, etc., as well as other applications are also contemplated. Device and method embodiments discussed herein may be used for pain medication, chemotherapy, iron chelation, immunoglobulin treatment, dextrose or saline IV delivery, treatment of various conditions including, e.g., pulmonary hypertension, or any other suitable indication or application. Non-medical applications are also contemplated.

With regards to the above detailed description, like reference numerals used therein may refer to like elements that may have the same or similar dimensions, materials, and configurations. While particular forms of embodiments have been illustrated and described, it will be apparent that various modifications can be made without departing from the spirit and scope of the embodiments herein. Accordingly, it is not intended that the invention be limited by the forgoing detailed description.

The entirety of each patent, patent application, publication, and document referenced herein is hereby incorporated by reference. Citation of the above patents, patent applications, publications and documents is not an admission that any of the foregoing is pertinent prior art, nor does it constitute any admission as to the contents or date of these documents.

Also incorporated herein by reference in their entirety are commonly owned U.S. Pat. Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,201,656; 10,279,105; 10,279,106; 10,279,107; 10,357,603; 10,357,606; 10,492,141; Ser. No. 10/541,987; U.S. Pat. Nos. 10,569,016; 10,736,037; 10,888,655; 10,994,077; 11,116,901; 11,224,693; 11,291,763; 11,305,057; 11,458,246; 11,464,908; and 11,654,236 and commonly owned U.S. Patent Publication Nos. 2009/0287180; 2012/0123230; 2013/0053816; 2014/0276423; 2014/0276569; 2014/0276570; 2018/0071454; 2019/0307952; 2020/0206420; 2020/0329433; 2020/0368430; 2020/0372995; 2021/0001044; 2021/0113766; 2021/0353857; 2022/0062553; 2022/0139522; 2022/0223250; 2022/0233772; 2022/0233773; 2022/0238201; 2022/0265927; 2023/0034408; 2022/0344017; 2022/0370708; 2022/0037465; 2023/0040677; 2023/0047034; 2023/0113545 and 2023/0113755 and commonly owned U.S. patent application Ser. Nos. 17/368,968; 17/896,492; 18/011,060; 18/071,814; 18/071,835; 18/075,029; 18/090,788 18/115,316; and 18/139,391.

Modifications may be made to the foregoing embodiments without departing from the basic aspects of the technology. Although the technology may have been described in substantial detail with reference to one or more specific embodiments, changes may be made to the embodiments specifically disclosed in this application, yet these modifications and improvements are within the scope and spirit of the technology. The technology illustratively described herein may suitably be practiced in the absence of any element(s) not specifically disclosed herein. The terms and expressions which have been employed are used as terms of description and not of limitation and use of such terms and expressions do not exclude any equivalents of the features shown and described or portions thereof and various modifications are possible within the scope of the technology claimed. Although the present technology has been specifically disclosed by representative embodiments and optional features, modification and variation of the concepts herein disclosed may be made, and such modifications and variations may be considered within the scope of this technology.

1. An ambulatory infusion pump, comprising:
a reservoir configured to contain a medicament;
a leadscrew having a threaded exterior surface extending through the reservoir; and
a syringe plunger disposed within the reservoir and having a threaded interior surface interfacing with the threaded exterior surface of the leadscrew such that rotation of the leadscrew causes linear movement of the syringe plunger through the reservoir to cause the medicament to be delivered from the reservoir to a user.

2. The ambulatory infusion pump of claim 1, wherein the syringe plunger has an outer perimeter shape generally matching a shape of an inner cross-section of the reservoir.

3. The ambulatory infusion pump of claim 2, wherein the syringe plunger provides a seal against the inner cross-section of the reservoir to prevent medicament from leaking proximally passed the syringe plunger.

4. The ambulatory infusion pump of claim 3, further comprising a sealing element extending around an interface between the syringe plunger and the inner cross-section of the reservoir.

5. The ambulatory infusion pump of claim 1, further comprising a sealing element disposed around an interface between the leadscrew and the syringe plunger.

6. The ambulatory infusion pump of claim 1, further comprising a motor configured to cause the leadscrew to rotate.

7. The ambulatory infusion pump of claim 6, wherein the syringe plunger does not rotate when the leadscrew rotates.

8. The ambulatory infusion pump of claim 6, wherein a portion of the leadscrew that interfaces with the motor is disposed outside of the reservoir.

9. The ambulatory infusion pump of claim 1, wherein the leadscrew is formed of a material that will not contaminate the medicament.

10. (canceled)

11. An ambulatory infusion pump, comprising:
a reservoir configured to contain a medicament;
a leadscrew extending through the reservoir; and
a syringe plunger disposed within the reservoir and having an opening through which the leadscrew is extended, wherein rotation of the leadscrew causes linear movement of the syringe plunger along the leadscrew through the reservoir to cause the medicament to be delivered from the reservoir to a user.

12. The ambulatory infusion pump of claim 11, wherein the syringe plunger has an outer perimeter shape generally matching a shape of an inner cross-section of the reservoir.

13. The ambulatory infusion pump of claim 12, wherein the syringe plunger provides a seal against the inner cross-section of the reservoir to prevent medicament from leaking proximally passed the syringe plunger.

14. The ambulatory infusion pump of claim 13, further comprising a sealing element extending around an interface between the syringe plunger and the inner cross-section of the reservoir.

15. The ambulatory infusion pump of claim 11, further comprising a sealing element disposed around an interface between the leadscrew and the syringe plunger.

16. The ambulatory infusion pump of claim 11, further comprising a motor configured to cause the leadscrew to rotate.

17. The ambulatory infusion pump of claim 16, wherein the syringe plunger does not rotate when the leadscrew rotates.

18. The ambulatory infusion pump of claim 16, wherein a portion of the leadscrew that interfaces with the motor is disposed outside of the reservoir.

19. The ambulatory infusion pump of claim 11, wherein the leadscrew is formed of a material that will not contaminate the medicament.

20. (canceled)

21. The ambulatory infusion pump of claim 6, further comprising a common housing containing the reservoir and the motor.

22. The ambulatory infusion pump of claim 16, further comprising a common housing containing the reservoir and the motor.
